(19) United States
(12) Patent Application Publication
(10) Pub. No.: US 2023/0071660 A1
(43) Pub. Date: Mar. 9, 2023

(54) DELIVERY OF MEDICATION TO A TREATMENT SITE WITHIN A BODY

(71) Applicant: InCube Labs, LLC, San Jose, CA (US)

(72) Inventors: Paul Spehr, San Antonio, TX (US); Kyle Horlen, Shavano Park, TX (US); Kristina Kaminskaya, San Jose, CA (US)

(21) Appl. No.: 17/799,843

(22) PCT Filed: Feb. 17, 2021
(86) PCT No.: PCT/US2021/018401

§ 371 Date: Aug. 15, 2022

Related U.S. Application Data
(60) Provisional application No. 62/977,842, filed on Feb. 18, 2020.

(51) Int. Cl. A61M 25/00; A61M 25/04; A61M 39/02

(52) U.S. Cl. CPC A61M 25/007; A61M 25/04; A61M 39/0247; A61M 2039/025; A61M 2039/0276; A61M 2039/0282; A61M 2210/0693

(57) ABSTRACT

The devices, systems, and methods include a catheter including a flexible tube having a proximal end and a distal end, the catheter defining a lumen extending from the proximal end to the distal end. The tube defines a plurality of slits. The distal end of the tube includes an atraumatic tip configured to be advanced through an opening in a skull to a target treatment site within the brain. A detachable connector is attached to the proximal end of the tube. Upon delivery of a fluid to the catheter from an external fluid source, the slits in the tube are responsive to pressure of the fluid within the catheter, by the slits opening to disperse the fluid from the lumen of the tube. The slits are responsive to a drop in pressure of the fluid within the catheter, by the slits closing.

### CROSS-REFERENCE TO RELATED APPLICATIONS

This application claims benefit of priority to U.S. Provisional Patent Application No. 62/977,842, filed on Feb. 18, 2020 and titled “Systems and Methods for Treatment of Glioblastoma”; the aforementioned priority application being hereby incorporated by reference in its entirety for all purposes.

### BACKGROUND

Many conditions are treated by delivering medication to a treatment site within a body. However, traditional approaches to drug delivery such as oral ingestion or intravascular injection may not be as efficacious as desired. For example, because only a small percentage of a delivered medication may reach the target treatment site due to distribution of the medication throughout the body by systemic blood circulation, dosages of the medication as delivered may need to be significantly increased so that an amount of the medication that reaches the treatment site is therapeutically effective. This increased dosage can lead to side effects in, and can further result in harm to, non-target areas of the body.

An example of a treatment site with respect to the brain is a glioblastoma, also known as glioblastoma multiforme, an aggressive cancer that begins within the brain and is the most common primary malignant brain tumor in adults, encompassing 16% of all primary brain and central nervous system neoplasms. Even with advanced diagnostic modalities and multidisciplinary treatments that include one or more of maximal surgical resection, radiotherapy, and chemotherapy, almost all patients experience tumor progression with nearly universal mortality due to the glioblastoma. The efficacy for treatment of glioblastoma multiforme and other brain cancers or tumors via traditional drug delivery systems is limited. Roughly 99% of administered chemotherapy drugs may not even reach the cancer or tumor site. Additionally, a drug's inability to cross the blood-brain barrier (a highly selective semipermeable border that separates the circulating blood from the brain and extracellular fluid in the central nervous system) may further limit efficacy.

More generally, traditional approaches of drug delivery can have limited efficacy in reaching a target treatment site within a body. Thus, there is a need for providing targeted delivery of medication to treat specific, localized tissues or anatomical locations. Further, a treatment plan can include delivery of such fluid continuously, periodically, or occasionally over a period of time; therefore it may be desirable to retain a delivery device within the body for a time at least sufficient to complete the treatment plan. It may further be desirable for the treatment device to seal when medication is not being delivered.

### SUMMARY

Embodiments of the present disclosure include improved systems and methods for providing targeted delivery of a therapeutic preparation to a treatment site in the body.

In an embodiment, the devices, systems, and methods for treatment within a brain, include a catheter including a flexible tube having a proximal end and a distal end, the catheter defining a lumen extending from the proximal end to the distal end. The tube defines a plurality of slits. The distal end of the tube includes an atraumatic tip configured to be advanced through an opening in a skull of a body to a target treatment site within the brain. A detachable connector is attached to the proximal end of the tube. Upon delivery of a fluid containing the therapeutic preparation to the catheter from an external fluid source, the slits in the tube are responsive to pressure of the fluid within the catheter such that the slits expand and open to disperse the fluid outward from the lumen of the tube. The slits are responsive to a drop in pressure of the fluid within the catheter, by the slits closing.

Further aspects of these and other embodiments are described more fully below, with reference to the drawings.

### BRIEF DESCRIPTION OF THE DRAWINGS

Embodiments described herein will be more fully understood by reference to the following drawings which are for illustrative purposes only:

FIG. 1A shows a schematic side view of a treatment device including a catheter, according to one or more embodiments.

FIG. 1B shows a schematic side view of a treatment device of FIG. 1A during delivery of a fluid to the catheter for delivery to a treatment site, according to one or more embodiments.

FIG. 2A shows a slice view of an embodiment of a catheter of FIG. 1A illustrating a single slit shown in a closed configuration.

FIG. 2B shows a slice view of an embodiment of a catheter of FIG. 1B illustrating a single slit shown in an open configuration.

FIG. 2C shows a slice view of an embodiment of a catheter of FIG. 1B having multiple slits disposed radially about a circumference of the catheter, the slits shown in an open configuration.

FIG. 3A shows an expanded side view of a distal tip of a catheter having a single linear array of slits, the slits shown in an open configuration, according to one or more embodiments.

FIG. 3B shows an expanded side view of a distal tip of a catheter having multiple slit arrays disposed radially about the circumference of the catheter, the slits shown in an open configuration, according to one or more embodiments.

FIG. 4A shows a schematic diagram of an embodiment of the catheter of FIG. 1A positioned at a treatment site in a brain of a patient.

FIG. 4B shows a schematic diagram of an embodiment of the catheter of FIG. 4A coupled to an external tubing and a pump, during delivery of fluid to the catheter.

FIG. 5 shows a side view of the distal end of a catheter positioned a treatment site of a patient, with moveable sleeves disposed on the catheter to focus delivery of fluid from the catheter, according to one or more embodiments.

FIG. 6 shows a side view of a distal end of a catheter positioned at a treatment site of a patient, with slidable discs disposed on the catheter to minimize migration of fluid along the catheter past the discs, and with an anchor disposed at a distal end of the catheter to secure the catheter at the treatment site, according to one or more embodiments.

FIG. 7 shows a slice view of an embodiment of the catheter of FIG. 6.

### DETAILED DESCRIPTION

Embodiments of the present technology provide devices, systems, and methods for delivering treatment/therapy to a treatment site within a patient, and particular embodiments include a catheter for intracranial delivery of a therapeutic preparation, for example, to a brain tumor such as a glioblastoma multiforme (GBM).

The devices, systems, and methods described herein provide for targeted delivery of a therapeutic preparation, to concentrate the therapeutic preparation at tissues of interest while reducing a relative concentration of the therapeutic preparation in other tissues. For example, by avoiding systemic circulation and thus inhibiting non-specific distribution in the liver and spleen and avoiding the host's defense mechanisms, the system and methods of the present description can reach a target treatment site in higher concentrations to improve efficacy, while reducing side effects involved with delivery of the therapeutic preparation to other regions in the body upon which such therapeutic preparation may have a deleterious effect.

While embodiments of the present description are particularly suited for providing treatment for brain cancers or tumors, it is appreciated that the systems and methods disclosed herein may be used on a variety of different conditions or anatomies, for example treatment of other intracranial tumors, conditions or anomalies, or treatment of other locations in the body where direct, localized application of a therapeutic preparation is desired for treatment.

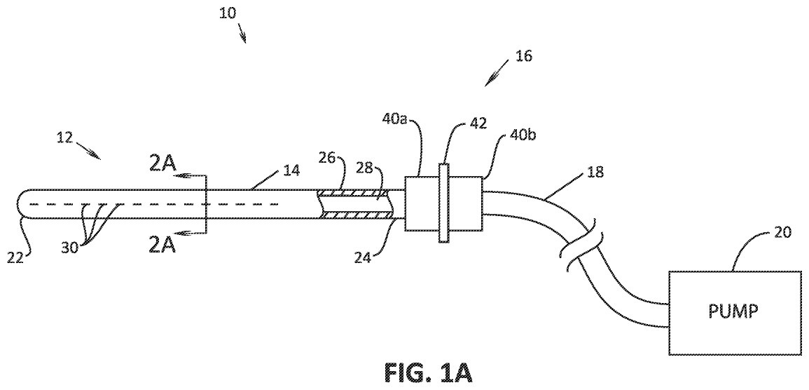

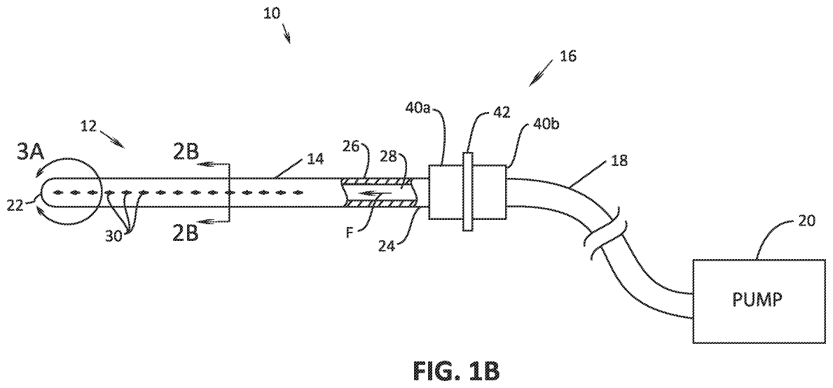

FIG. 1A illustrates an embodiment of a treatment device 10 for delivering a therapeutic preparation to a treatment site in the body. Treatment device 10 includes a drug-eluting catheter 12. Catheter 12 includes an infusion member in the form of a tube 14. Tube 14 has a distal end 22, and a proximal end 24 terminating at a detachable connector 16. Proximal end 24 is shown with a cutout portion to reveal an inner structure including a wall 26 of tube 14 and an interior lumen 28 of tube 14. Connector 16 provides a releasable attachment between tube 14 and an external tubing 18. Tube 14 may be flexible along its length or flexible along at least a portion of its length including near distal end 22.

In an embodiment, connector 16 includes a first coupling member 40a that is coupled to proximal end 24 of tube 14, a second coupling member 40b that is coupled to a distal end of external tubing 18, and a flange 42 between first coupling member 40a and second coupling member 40b. External tubing 18 is coupled at its proximal end to a fluid source and/or a pump, collectively illustrated and referred to for convenience herein as a pump 20.

In an embodiment, tubing 18 and pump 20 are coupled to catheter 12 by way of detachable connector 16 prior to a treatment period, decoupled after the treatment period is completed, and coupled again for a subsequent treatment period if applicable. In other embodiments, tubing 18 remains coupled to catheter 12 by way of detachable connector 16 after initial placement until later such as when catheter 12 is removed, and pump 20 is coupled to tubing 18 prior to a treatment period, decoupled after the treatment period is completed, and coupled again for a subsequent treatment period if applicable. In yet further embodiments, tubing 18 and pump 20 both remain coupled to catheter 12 by way of detachable connector 16 after initial placement until later removed such as until catheter 12 is removed.

Catheter 12 is positioned within a body. Tubing 18 may be external to the body or may be implanted in the body. Pump 20 may be external to the body or may be implanted in the body. Connector 16 may be external to the body or partially within the body; for example, first coupling member 40a may be positioned within the body, second coupling member 40b may be positioned external to the body, and flange 42 may be positioned adjacent to skin of the body inside (under the skin) or outside (over the skin) of the body. The releasable coupling of connector 16 (which may include, for example, a lure lock or other releasable fastening device) may allow catheter 12 to remain in a patient at or near a surface of the patient's body (e.g., at or near a surface of the patient's skull) while other portions of treatment device 10 (e.g., pump 20, external tubing 18, and all/or a portion of detachable connector 16) are detached until treatment is to be provided.

In some embodiments, pump 20 may be configured to be implanted in the patient (e.g., at the base of the neck, or in the back or pectoral area). In other embodiments, pump 20 may be worn by the patient (e.g., on a belt, shoulder strap or the like). In particular embodiments, pump 20 corresponds to a miniature infusion pump known in the medical infusion arts (such as a wearable or implantable insulin pump) allowing for the pump to be readily implantable or easily worn or attached externally to the patient or clothing. In one particular embodiment, pump 20 incorporates a Synchromed II pump available from the Medtronic Corporation.

Pump 20 may correspond to a variety of medical pumps known in the medical arts including, for example, displacement pumps (e.g., a piston pump), peristaltic pumps, screw pumps and like devices. In an embodiment, pump 20 can be miniaturized for implantation, such as in the base of the head or neck area of the patient or other portion of the body. Miniaturized pumps for use as pump 20 may include MEMs and/or bubble jet based miniature pumps.

Pump 20 may be programmable via external buttons/switches (not shown) or operably coupled to an internal controller (not shown) including a microprocessor circuitry, logic and application programming executable thereon, wherein the controller may also be accessed and programmed by an external device (not shown) such as a cell phone, tablet, laptop, or other computing device. The programmability of pump 20 circuitry and associated application programming may include one or more routines allowing for control of one or more of flow rate, total volume delivered, and pump pressure.

Pump 20 may include circuitry/programming for detecting and signaling alarms when pump 20 detects one or more of the following: blockages in the flow path, air in the flow path inside the pump, when a selected volume of solution has been delivered, when an internal or external drug reservoir is almost empty or is empty, or other situation that is desirable to be notified.

Pump 20 contains a drug reservoir (not shown) and/or is configured to be coupled to an external drug reservoir (not shown). In an embodiment, pump 20 is configured to be coupled to a drip bag such as, or similar to, an IV drip bag.

Pump 20 may be configured to pump at low flow rates (e.g., in the 1-50 μl/min range) and/or low pressures, and vary said rates and/or pressures according to a desired treatment protocol. Pump 20 pumps fluid from a reservoir into external tubing 18 and correspondingly into tube 14 of catheter 12.

The term “fluid” as used herein refers to any solution that exhibits fluidic properties or can otherwise be pumped by pump 20 from a reservoir into external tubing 18 and correspondingly into tube 14 of catheter 12. Thus, a fluid may be, for example, in the form of a gas, a liquid, a colloidal suspension, a gel, a slurry, or a powder. A fluid can include a therapeutic preparation, a cleansing preparation, a hydrating preparation, or other preparation or a combination of preparations.

The term “therapeutic preparation” refers herein to a preparation including one or more components where the preparation is intended for a therapeutic, diagnostic, or other biological purpose. Each therapeutic preparation can include one or more components. A component of a therapeutic preparation can be, for example, a pharmacologically active agent, a DNA or SiRNA transcript, a cell, a cytotoxic agent, a vaccine or other prophylactic agent, a nutraceutical agent, a vasodilator, a vasoconstrictor, a delivery enhancing agent, a delay agent, an excipient, a diagnostic agent, or a substance for cosmetic enhancement.

A pharmacologically active agent can be, for example, an antibiotic, a nonsteroidal anti-inflammatory drug (NSAID), an angiogenesis inhibitor, a neuroprotective agent, a chemotherapeutic agent, a peptide, a protein, an immunoglobulin (e.g., a TNF-alpha antibody), an interleukin in the IL-17 family of interleukins, an anti-eosinophil antibody, another antibody, a nanobody, a large molecule, a small molecule, or a hormone, or a biologically active variant or derivative of any of the foregoing.

A cell can be, for example, a stem cell, a red blood cell, a white blood cell, a neuron, or other viable cell. Cells can be produced by or from living organisms or contain components of living organisms. A cell can be allogeneic or autologous.

A vaccine can be, for example, against an influenza, a coronavirus, meningitis, human papillomavirus (HPV), or chicken pox. A vaccine can correspond to an attenuated virus.

A nutraceutical agent can be, for example, vitamin A, thiamin, niacin, riboflavin, vitamin B-6, vitamin B-12, another B-vitamin, vitamin C (ascorbic acid), vitamin D, vitamin E, folic acid, phosphorous, iron, calcium, or magnesium.

A vasodilator can be, for example, 1-arginine, sildenafil, a nitrate (e.g., nitroglycerin), or epinephrine.

A vasoconstrictor can be, for example, a stimulant, an amphetamine, an antihistamine, epinephrine, or cocaine.

A delivery enhancement agent can be, for example, a permeation enhancer, an enzyme blocker, a peptide that permeates through mucosa, an antiviral drug such as a protease inhibitor, a disintegrant, a superdisintegrant, a pH modifier, a surfactant, a bile salt, a fatty acid, a chelating agent, or a chitosan. A delivery enhancing agent can, for example, serve as a delivery medium for delivery of a component of a therapeutic preparation, or serve to improve absorption of a component of a therapeutic preparation into the body. For example, a delivery enhancing agent can prime an epithelium of the intestine (e.g., fluidize an outer layer of cells) to improve absorption and/or bioavailability of one or more other components included in the therapeutic preparation.

A delay agent can be, for example, poly(lactic acid) (PLA), poly(glycolic acid) (PGA), polyethylene glycol (PEG), poly(ethylene oxide) (PEO), poly (I-lactic acid) (PLLA), poly(D-lactic acid) (PDLA), another polymer, or a hydrogel. A delay agent can be included with (e.g., mixed with, or providing a structure around) one or more other component(s) in a therapeutic preparation to slow a release rate of the other component(s) from the therapeutic preparation.

An excipient can be, for example, a binder, a disintegrant, a superdisintegrant, a buffering agent, an anti-oxidant, or a preservative. Excipients can provide a medium for a component of a therapeutic preparation (e.g., for assisting in manufacture), or to preserve integrity of a component of a therapeutic preparation (e.g., during manufacture, during storage, or after delivery prior to dispersion at a target treatment site).

A diagnostic agent can be, for example, a sensing agent, a contrast agent, a radionuclide, a fluorescent substance, a luminescent substance, a radiopaque substance, or a magnetic substance.

The configuration for pump 20 (e.g., flow rate, pressure) can be adjusted based on characteristics of the fluid to be pumped into external tubing 18 and thus into tube 14 of catheter 12. Tube 14 defines one or more slits 30 to allow the pumped fluid to exit tube 14 and be delivered to a treatment site. Slits 30 are formed or cut along at least a portion of a length of tube 14, particularly at or near distal end 22. Size and shape of individual slits 30 may be consistent within a manufacturing tolerance, or may vary. In the embodiment of FIG. 1A, slits 30 are arranged in a single linear array extending longitudinally along a portion of the length of tube 14; other arrangements of slits 30 are within the scope of the present disclosure. Slits 30 extend through an exterior of wall 26 of tube 14 to an interior of lumen 28 of tube 14.

Slits 30 are configured such that when tube 14 is not pressurized (e.g., when fluid is not being delivered to catheter 12, or when pressure of fluid in tube 14 drops below a threshold defined by a material of tube 14 and/or a structure of tube 14 and slits 30), slits 30 are in a closed configuration that maintains a seal or barrier between lumen 28 and an environment exterior to tube 14. FIG. 1A illustrates treatment device 10 in a configuration in which slits 30 are closed and fluid is not being delivered to the treatment site.

Fluid that is pumped into lumen 28 of catheter 12 from proximal end 24 of tube 14 flows to distal end 22. As a result of continued delivery of fluid into lumen 28, pressure builds within lumen 28 and thus pressure is exerted against an interior surface of wall 26 of tube 14.

FIG. 1B illustrates an embodiment of treatment device 10 in a configuration in which a fluid F is flowing into catheter 12 such that slits 30 are opened by pressure of fluid F within lumen 28, and fluid F can be delivered through open slits 30 to the treatment site. Slits 30 are responsive to the pressure against wall 26 such that slits 30 expand from their closed configuration to their open configuration when the pressure reaches or exceeds a threshold defined by the design of, including the materials used in, tube 14. The opening of slits 30 allows fluid F to be dispersed out of tube 14 and directed into surrounding tissue. Size and shape of slits 30 may be designed or modified to accommodate characteristics of fluid F.

In an embodiment, tube 14 includes a flexible biocompatible polymer or plastic, such as silicone, polyurethane, PVC (polyvinyl chloride), PEBAX or other flexible material. The flexibility may be adjusted with a shape and dimensions of tube 14 and/or by a specific material selection (e.g., having a selected durometer, elasticity, or other parameter). The flexibility allows slits 30 to open when pressure in lumen 28 is at or above a threshold pressure and to automatically self-seal or close when pressure in lumen 28 subsides to or below the threshold pressure. The flexibility may be configured to allow distal end 22 to be guided/navigated through circuitous or tortuous anatomy if applicable and/or desirable, such as through the various lobes or ventricles of the brain.

While the embodiment illustrated in FIG. 1A and FIG. 1B is shown having a single linear array of slits 30, tube 14 may include other arrangements of slits 30. For example, slits 30 may be arranged in one or more arrays radially spaced apart in addition to or alternative to one or more arrays with axial/longitudinal spacing as shown in FIG. 1A.

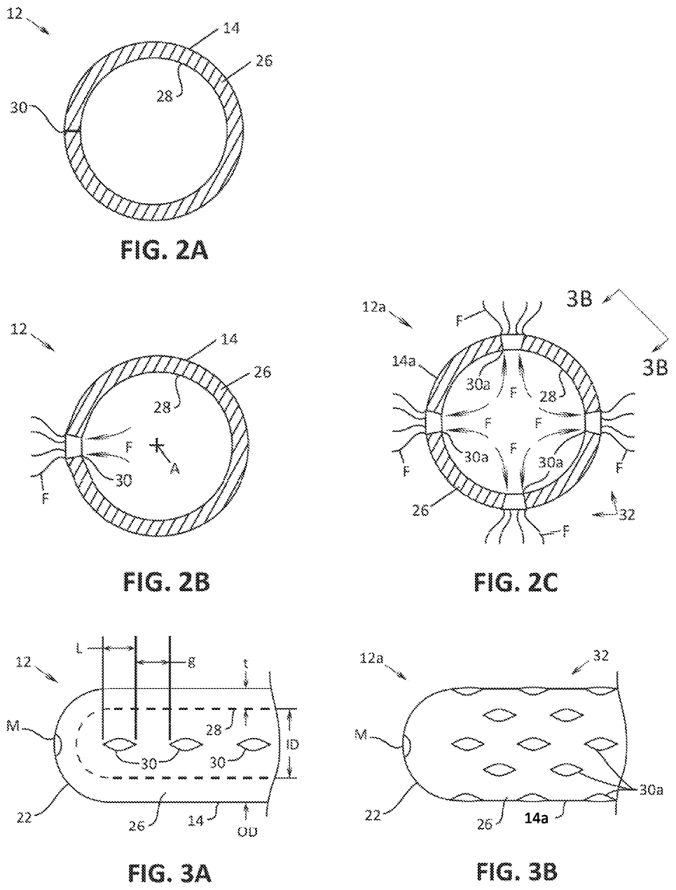

FIG. 2A illustrates a slice view of an embodiment of catheter 12 along line 2A of FIG. 1A, illustrating one of the slits 30 in a closed configuration. FIG. 2B is a slice view of an embodiment of catheter 12 along line 2B of FIG. 1B, illustrating one of the slits 30 in an open configuration.

FIG. 2C illustrates a slice view of a catheter 12a (an embodiment of catheter 12) with multiple radial slits 30a designed to disperse fluid F from multiple locations along a circumference of a tube 14a (an embodiment of tube 14). While the embodiment shown in FIG. 2C shows a radial array 32 of four slits 30a dispersed at approximately a 90° spacing with respect to each other along the circumference, it is appreciated that any number of slits or increments may be selected (e.g. three slits at 120°, six slits at 60°, two or more slits within 70°, or slits randomly spaced around the circumference).

FIG. 3A is an expanded view of an embodiment of distal end 22 of catheter 12 having a single linear array of slits 30, shown in an open configuration (i.e., catheter 12 is pressurized with fluid F to open slits 30 for dispersion of fluid F to the treatment site).

FIG. 3B is an expanded view of an embodiment of distal end 22 of catheter 12a having a radial array 32 of multiple linear arrays of slits 30a in an alternating radially-staggered configuration, where adjacent sets of the 4×90° pattern shown in the section view of FIG. 2C are radially offset by 45°. Such spacing provides a distributed emission pattern of fluid F both axially and radially along an outer surface of tube 14a.

In general, placement and spacing of slits (e.g., slits 30 or slits 30a) may not be symmetric or may not be arranged in a pattern. For example, slits 30 or slits 30a may be disposed randomly on tube 14 or tube 14a, respectively. For another example, slits 30a may be arranged to dispense fluid F from only one side of tube 14a (e.g. slits 30a may extend over 180° of the circumference of tube 14a) or slits 30a may be arranged so that they dispense fluid F from a portion of a side of tube 14a (e.g., within a 40° portion of the circumference of tube 14a).

The dimensions of catheter 12 (or 12a) may be varied according to the specified treatment modality and tissue to be treated. FIG. 3A includes various dimensioning of catheter 12 for reference, which dimensioning is also applicable to catheter 12a. Dimensioning shown includes an outer diameter (OD) of tube 14/14a, an inner diameter (ID) of tube 14/14a, a thickness t of wall 26, an axial length L of slits 30, and a gap g between slits 30. In an embodiment, tube 14/14a of catheter 12/12a has an OD in a range of 1.00 mm to 2.00 mm, an ID in a range of 0.25 mm to 1.00 mm, and a wall 26 thickness tin a range of 0.25 mm to 0.75 mm. In an embodiment, slits 30/30a have an axial length L in a range of 0.25 mm to 1.00 mm. In an embodiment, slits 30/30a are spaced apart according to a gap g in a range of 0.50 mm-5.00 mm. The above dimensions are provided by way of example only, and differing dimensions and spacing may be implemented according to a desired or intended application.

Catheter 12 and catheter 12a as shown in FIG. 3A and FIG. 3B, respectively, each have a blunt (e.g., hemispherical) atraumatic distal end 22 to promote advancement of catheter 12/12a through the patient's anatomy without disruption to healthy tissue or anatomy while travelling to a target treatment site. In other embodiments, distal end 22 may be designed with a variety of shapes to serve this purpose.

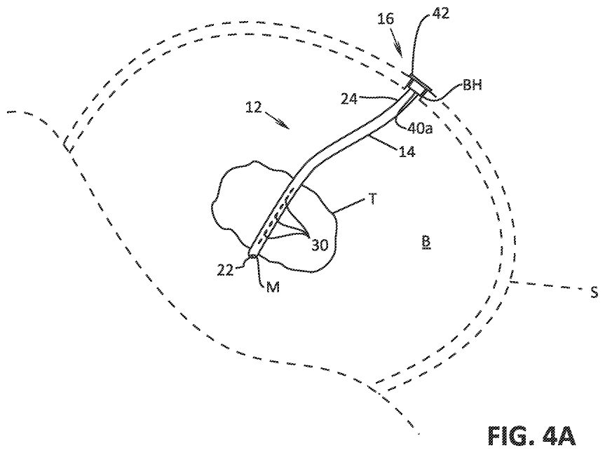

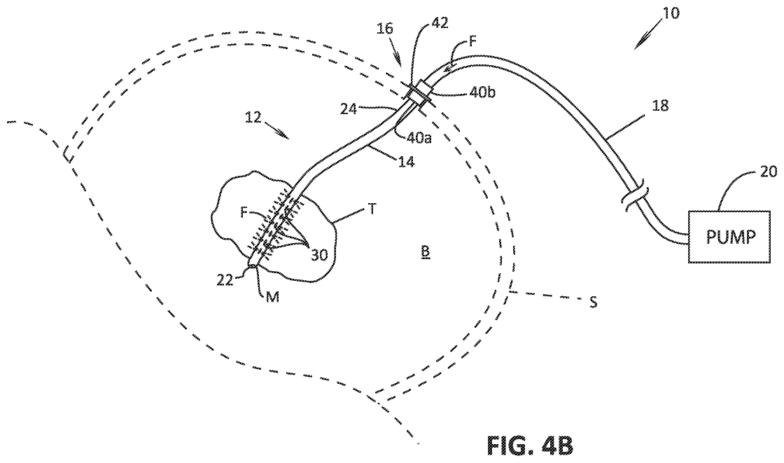

Catheter 12/12a may include one or more markers at or near distal end 22, such as radiopaque marker M in FIGS. 3A, 3B, 4A, 4B, and/or along catheter 12/12a. In this way, a present location of distal end 22 may be readily identified during or after delivery and placement of catheter 12/12a to the target treatment site (e.g., via radiographic or fluoroscopic guidance).

FIG. 4A illustrates catheter 12 as delivered to a treatment site in a brain B of a patient in an embodiment. In one method of introduction for intracranial treatment (e.g., for treatment of GBM), after initial surgical preparation/incision, a burr hole BH is generated with an air drill or other instrument through a skull S of the patient. A burr hole plug (not shown) may be placed at the location of burr hole BH, such as for ease of coupling to connector 16 or for maintaining catheter 12 in a treatment position. Distal end 22 of tube 14 of catheter 12 is fed through burr hole BH and advanced into brain B to a target treatment site, shown in FIG. 4A as a tumor T for convenience and without limitation. Stereotactic and/or other surgical techniques such as use of a stylet may be used to help advance tube 14 within/between ventricles to the target treatment site. As shown in FIG. 4A, distal end 22 of catheter 12 may be advanced into (or past) the target treatment site so as to position slits 30 for delivering fluid F. Radiopaque marker M may be used to position distal end 22 with respect to the target treatment site and surrounding anatomy via radioscopic or fluoroscopic guidance. A craniotomy may also be performed to provide additional visual and/or manual guidance of catheter 12. First coupling member 40a of connector 16 is positioned in or at the entry of burr hole BH, and may rest flush with an outer surface of the skull S or seat at the opening in the skull S defined by burr hole BH. Burr hole BH and first coupling member 40a may be sized or shaped so that first coupling member 40a is retained at its location within the skull (e.g., with the diameter of first coupling member 40a tapered or sized for press fit with skull S). Flange 42 is positioned adjacent to first coupling member 40a and over the skull S (above or below the skin) to inhibit motion of first coupling member 40a and connector 16, and to protect and/or seal burr hole BH from the external environment. An internal flange (not shown), may also be provided on first coupling member 40a such that skull S is positioned between flange 42 and the internal flange, for further motion inhibition and protection/sealing. Flange 42 and/or the internal flange (if applicable) may be attached to skull S by screws or other retention mechanisms, by adhesives, or by sealants. In an embodiment, catheter 12 and detachable connector 16 (or a portion thereof such as first coupling member 40a and optional internal flange) are maintained within skull S for a length of time (e.g., hours, days, weeks, months, years). Although not shown in FIG. 4A, second coupling member 40b may be disposed adjacent to flange 42. Second coupling member 40b may be attached to or formed with first coupling member 40a and/or flange 42. In an embodiment, second coupling member 40b is attached to or formed with external tubing 18.

FIG. 4B illustrates treatment device 10 in a configuration for providing treatment in an embodiment. Second coupling member 40b is coupled to first coupling member 40a of catheter 12 to form connector 16 for coupling external tubing 18 and catheter 12. Pump 20 is connected and can be operated to deliver fluid F to catheter 12 according to a specified treatment protocol (e.g., at a specified delivery rate for a specified time, once or periodically, or periodically with varying delivery rates and/or times). An example of a delivery rate for fluid F is 1-50 μl/s.

In an embodiment, external tubing 18 and pump 20 are coupled to catheter 12 by way of detachable connector 16 to provide treatment and then are detached; in other embodiments, external tubing 18 (with or without pump 20) is coupled by way of detachable connector 16 to catheter 12 whether or not treatment is being provided. External tubing 18 and/or pump 20 may be implanted in the patient's body whether or not connected to catheter 12.

Slits 30 operate as a one-way valve to allow fluid F to flow out of lumen 28 of tube 14 and diffuse and infuse into tissue at the treatment site (e.g., tissue of tumor T). After flow from pump 20 has stopped, the flow of fluid F and associated pressure inside lumen 28 decreases until slits 30 close and seal. In the closed configuration, slits 30 inhibit ingress of body fluids, materials or tissue growth into lumen 28. In an embodiment, an amount of non-therapeutic fluid (e.g., saline) equal to a total internal fluidic volume of external tubing 18, detachable connector 16, and catheter 12 combined is pumped into external tubing 18 to force a therapeutic treatment fluid F completely out of catheter 12 so that the therapeutic treatment fluid F does not remain within catheter 12 after treatment.

In an embodiment, once a treatment period is finished, external tubing 18 and pump 20 may be removed (e.g., by detaching second coupling member 40b from first coupling member 40a), and catheter 12 may be retained in place for a later treatment. The catheter may be closed at first coupling member 40a via a cap (not shown).

Slits 30 may be positioned and oriented relative to the location of the target treatment site as desirable for a treatment plan. For example, catheter 12 having slits 30 along a single side of tube 14 may be positioned at one side of the target treatment site so that slits 30 provide fluid F toward the target treatment site. For another example, catheter 12 having slits 30 at various radial positions around the circumference of tube 14 may be positioned within a target treatment site so that slits 30 provide fluid F to a treatment area around catheter 12. Tube 14 with any pattern of slits 30 may be positioned adjacent to or within a target treatment site. Tube 14 may also be repositioned (e.g., translated and/or rotated) to treat other sections of the target treatment site. In an embodiment, tube 14 may be centrally located within the target treatment site, and tube 14 is rotated about its axis (e.g., axis A in FIG. 2B) to sweep across a treatment zone.

In various embodiments, catheter 12 is provided in a standardized form. In an embodiment, slits 30 are positioned in an individualized pattern on tube 14 to provide dispersion of fluid F across an asymmetrically-shaped target treatment site of a particular patient. In an embodiment, a number of slits 30 is selected to treat a specific condition for a specific patient.

Catheter 12 may include additional features to allow for further refinement of fluid F delivery. In an embodiment, one or more moveable sleeves (e.g., tubes) are applied to an outer surface of catheter 12 to focus delivery of fluid F. The sleeves may be sized and positioned to cover selected ones of slits 30 that would, if allowed to open, disperse fluid F to non-target (e.g., healthy tissue) regions in proximity to the target treatment site. Any number of sleeves may be employed, and may include windows or other shapes for directional application of radial fluid emission.

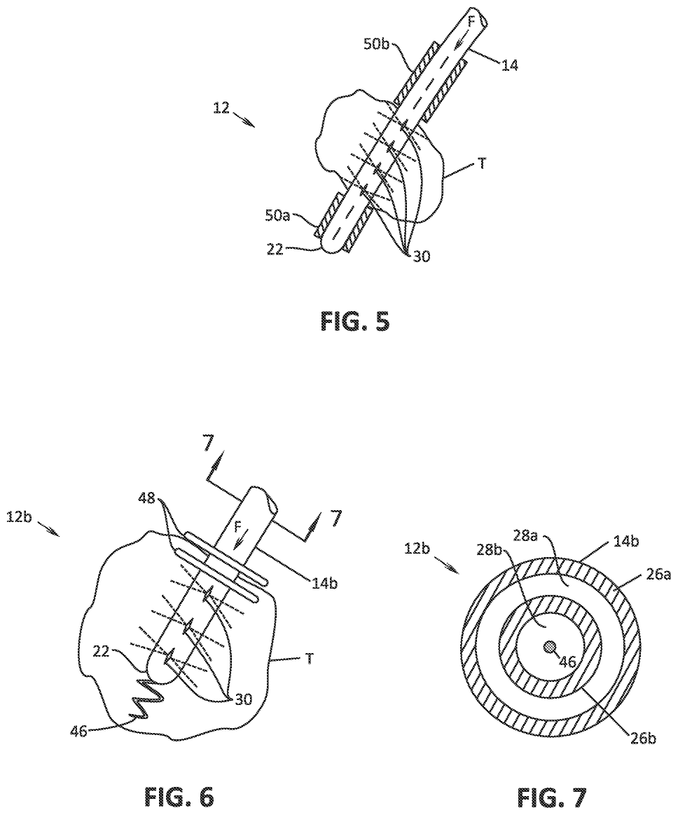

FIG. 5 illustrates an embodiment of distal end 22 of catheter 12 (shown with a pattern of slits 30 at least similar to that in FIG. 1A) at a treatment site shown as tumor T for convenience. In this embodiment, a sleeve 50a and sleeve 50b are disposed over tube 14 and cover various of slits 30 that are outside the boundary of tumor T so that delivery of fluid F may be limited to the confines of tumor T. Sleeve 50a and sleeve 50b have an inner diameter that closely matches the OD of tube 14, such that expansion of slits 30 within the confines of sleeves 50a/50b is restricted and slits 30 are prevented from fully opening.

Catheter 12 having tube 14 in FIG. 4A, FIG. 4B, or FIG. 5 may be replaced by another catheter for delivering fluid F to a treatment site, such as replacement by catheter 12a having tube 14a (FIG. 2C and FIG. 3B), catheter 12b having tube 14b (FIG. 6 and FIG. 7), or other catheter.

FIG. 6 illustrates an embodiment of distal end 22 of a catheter 12b (an embodiment of catheter 12) at a treatment site shown as tumor T for convenience. Catheter 12b includes additional features in the form of slidable discs 48 applied to an exterior of tube 14b to minimize back migration of delivered fluid F along catheter 12b.

Catheter 12b may also include an extendable anchor 46 that is configured to extend from distal end 22 of catheter 12b to engage tissue to secure location of catheter 12b at the treatment site. In an embodiment, anchor 46 has a helical shape. In other embodiments, anchor 46 has a non-helical shape. Attachment mechanisms other than anchor 46 (e.g. hooks, barbs, or clamps) may additionally or alternatively be employed. Although shown extending outwards approximately along a long axis of tube 14b of catheter 12b, anchor 46 could instead extend at an angle to the long axis of tube 14b. Further, although shown extending from a tip of tube 14b, anchor 46 could instead extend from a side of tube 14b. In an embodiment, catheter 12b includes two or more anchors 46 that extend at different angles from tube 14b and/or from different positions on tube 14b.

Attachment mechanisms (e.g., one or more anchor(s) 46 and/or one or more other attachment mechanisms) may be disposed within catheter 12b prior to deployment such that the attachment mechanism(s) do not protrude beyond a perimeter of tube 14b; the attachment mechanism(s) may be rotated or otherwise moved to translate the attachment mechanism(s) from a retracted position within tube 14b to the extended position shown in FIG. 6 to provide positive fixation (shown in FIG. 6 as fixation within tumor T). In an embodiment of a method of treatment, anchor 46 and/or other attachment mechanisms are retained inside tube 14b while catheter 12b is being delivered to a target treatment site; once at the target treatment site, the attachment mechanism(s) may be partially deployed (e.g., to expose a sharp distal tip of an attachment mechanism) to pierce the target treatment site and effect final advancement of distal end 22 into the target treatment site until distal end 22 is at a desired location, upon which further extension of the attachment mechanism(s) is effected to affix distal end 22 into the tissue. In another embodiment, the attachment mechanism(s) are retained within tube 14 until catheter 12b is positioned at the target treatment site and then are extended.

FIG. 7 is a slice view of an embodiment of catheter 12b along line 7 in FIG. 6. In this embodiment, catheter 12b includes a multi-channel lumen having an outer annular lumen 28a for delivery of fluid F to slits 30, and an inner concentric lumen 28b that allows for disposition of, and translation/rotation of, one or more attachment mechanisms such as anchor 46.

Introduction of a catheter such as catheter 12, catheter 12a, or catheter 12b may be performed by a doctor or medical professional, often through the use of an imaging modality (e.g., fluoroscopy, CT, MRI, ultrasound, endoscopic image capture, or other imaging technique) or sensing devices that allow attending personnel to position the treatment section of the catheter relatively precisely at the target treatment site. In an embodiment, a delivery process involves use of a separate delivery device (e.g., an introducer or a trocar) which can provide a conduit or path for the catheter as it is introduced. Once the catheter is positioned at the target treatment site, the delivery device can be removed.

In an embodiment, fluid F may be delivered for an extended period of time (e.g., hours or days). In an embodiment, fluid F is delivered for 96 hours, delivery is ceased for a period of time (e.g., one week or one month), and then delivery is repeated periodically or occasionally until treatment is completed. Treatment may be complete, for example, after a treatment regimen is finished, or after a tumor has regressed.

The delivered fluid F will generally include one or more therapeutic preparations configured to provide treatment to the treatment site. The therapeutic preparation may be a component at a specific concentration within fluid F, in addition to delivery vehicles for the therapeutic preparation. One of, or a combination of, the following types of delivery vehicles may be included within fluid F: polymeric micelles, liposomes, lipoprotein-based drug carriers, nano-particle drug carriers, and dendrimers. In preferred embodiments, the delivery vehicle(s) is/are non-toxic, biocompatible, non-immunogenic, biodegradable, and avoiding of recognition by the patient's defense mechanism.

In an embodiment, the therapeutic preparation may employ a nanoparticle-mediated drug delivery configuration. In such embodiments, the nanoparticles may be coated with a coating. For example, the nanoparticles may be coated with a polymer. In an embodiment, a nanoparticle coating includes polyethylene glycol (PEG), rendering the nanoparticle hydrophilic, thus allowing water molecules to bind to the oxygen molecules on the PEG via hydrogen bonding. The result of this bond is a film of hydration around the nanoparticle which makes the substance antiphagocytic. Thus, the drug-loaded nanoparticle is able to stay in circulation for a longer period of time than an uncoated nanoparticle.

For use in treating intracranial brain tumors such as GMB, fluid F may include one or more therapeutic preparations to slow, stop, reverse, or otherwise inhibit growth or proliferation of the tumor. Examples of such therapeutic preparations include chemotherapy, monoclonal antibodies, cytotoxins, immunotherapy, blockers (e.g., lactate blockers), and inhibitors.

Examples of chemotherapy include temozolomide and camptothecin or an analogue thereof (e.g., topotecan, irinotecan,10,11-methylenedioxy-CPT (MDC), and the alkylating derivative, 7-chloromethyl-10,11-MD).

Examples of cytotoxins include I-TM-601, transferrin-CRM107, interleukin (IL)-13, (IL)-13-PE38QQR, TP-38, and DAB389EGF.

Examples of immunotherapy include cytokines such as TGF-β2, SB-431542, AP12009, and IL-4.

Examples of inhibitors include tyrosine kinase inhibitors, angiogenesis inhibitors, and inhibitors of Ras/MAPK and PI3K/Akt pathways. Further examples of inhibitors include: avβ3 and avβ5 integrin inhibitors (Cilengitide); epidermal growth factor receptor (EGFR) inhibitors (e.g., Gefitinib, Erlotinib, Lapatinib(EGFR and ErbB-2 inhibitor), AEE788 (EGFR and VEGFR inhibitor), ZD6474 (VEGFR and EGFR inhibitor), EKB569, Cetuximab (anti-EGFR monoclonal antibody)); histone deacetylase inhibitors (e.g., Depsipeptide (FK228), Suberoylanilide hydroxamic acid (SAHA)); farnesyltransferase inhibitors (e.g., Tipifarnib, Lonafarnib); mammalian target of rapamycin (mTOR) inhibitors (e.g., Temsirolimus, Everolimus, Sirolimus, AP23573); platelet-derived growth factor receptor (PDGFR) inhibitors (e.g., Imatinib mesylate, PTK787 (PDGFR, VEGFR inhibitor), SU011248 (PDGFR, VEGFR, c-Kit inhibitor) Raf kinase inhibitors, Sorafenib (VEGFR, PDGFR, and Raf kinase inhibitor)); heat shock protein (HSP)-90 inhibitors (e.g., 17-AAG (17-allylamino-geldanamycin)); vascular endothelial growth factor receptor (VEGFR) inhibitors (e.g., Valatanib (PTK787) (PDGFR and VEGFR inhibitor), Sorafenib (VEGFR, PDGFR, and Raf kinase inhibitor), Sonitinib (PDGFR, c-Kit, and VEGFR inhibitor), AEE788, AZD2171, ZD6474 (VEGFR and EGFR inhibitor)); protein kinase C (PKC) inhibitors (e.g., Tamoxifen, Enzastaurin (PKC-82 inhibitor)); and proteasome inhibitors (e.g., Bortezomib).

In an embodiment, the target treatment site is an area of neuronal or glial cells, blood vessels, or cells of the immune system. A therapeutic preparation is formulated to amplify or attenuate neurotransmission to modulate sentience (e.g., depression), motor function (e.g., Parkinson's disease), or cognitive processing (e.g., schizophrenia), formulated to chemically or biologically ablate aberrant pathways (e.g., eleptogenic foci), or formulated to impact senescence (e.g., dementias including Alzheimer's disease).

As will be apparent from the description and drawings of the present disclosure, embodiments include without limitation:

An apparatus for targeted delivery of a therapeutic preparation in a body of a patient includes a catheter including a flexible tube having a proximal end and a distal end, the catheter defining a lumen extending from the proximal end to the distal end. The tube defines slits. The distal end of the tube includes an atraumatic tip configured to be advanced through an opening in the body of the patient to a target treatment site. The apparatus further includes a detachable connector including a first coupling member attached to the proximal end of the tube. Upon delivery of a fluid containing the therapeutic preparation to the catheter from an external fluid source, the slits in the tube are responsive to pressure of the fluid within the catheter such that the slits expand and open to disperse the fluid radially outward from the lumen of the tube. The slits are responsive to a drop in pressure of the fluid within the catheter such that the slits close.

A system for targeted delivery of a fluid to a body of a patient includes a catheter, an external tubing, and a fluid source. The catheter includes a flexible tube having a proximal end and a distal end, the catheter defining a lumen extending from the proximal end to the distal end. The tube defines slits, and the tube includes an atraumatic tip configured to be advanced to a treatment site within the body. The fluid source is configured for delivering a fluid into the catheter via the external tubing. Upon delivery of the fluid to the catheter, the slits are responsive to an applied pressure of the fluid within the catheter such that the slits expand and open to disperse the fluid radially outward from the tube. The slits are responsive to a drop in pressure within the catheter such that the slits close to self-seal the lumen.

A method for targeted delivery of a therapeutic preparation to a body of a patient includes generating an opening through a surface of the patient, delivering a catheter through the opening, and maneuvering the catheter until it is positioned at a target treatment site in the body. The catheter includes a flexible tube having a proximal end and a distal end, the catheter defining a lumen extending from the proximal end to the distal end of the tube. The distal end of the tube includes an atraumatic tip configured to allow advancement of the tube into the body, and the tube defines slits. The method further includes; operating a pump to deliver a fluid containing the therapeutic preparation into the catheter and thereby cause the slits to expand in response to an applied pressure within the catheter from the delivered fluid, the expansion of the slits opening the slits to allow dispensing of the fluid from the lumen through the slits; and ceasing delivery of the fluid to the catheter after a period of time, thereby causing a drop in pressure within the catheter such that the slits close and self-seal the tube.

Any of the apparatus, system, or method above, with any one of, or a combination of, the following features:
the slits are arranged in linear arrays oriented at radially spaced-apart locations along the circumference of the tube to disperse the fluid at multiple radial locations from the tube
external tubing having a distal end configured for detachably coupling at a/the detachable connector and a proximal end configured to be coupled to a fluid source
a/the detachable connector provides a sealable opening
one or more sleeves configured to be advanced over the tube, each of which has an inner diameter closely matching an outer diameter of the tube such that when the sleeve is advanced over one or more of the slits, those slits disposed under the sleeve are constrained from opening, thus focusing dispersion of fluid through the remainder of the slits which are not constrained by the sleeve
a deployable anchor configured to extend from a location within the tube; upon extension, the anchor engages tissue for fixation of the tube at the treatment site
one or more discs disposed over the tube, where the discs act to block dispersion of the fluid along the tube past the discs
a therapeutic preparation disposed in a/the fluid is provided through the catheter
operating a pump to deliver a/the fluid containing a/the therapeutic preparation into the catheter includes attaching an/the external tubing to the pump and to a/the detachable connector coupled to the catheter, then detaching the external tubing from the detachable connector and covering an opening of the detachable connector at completion of the treatment
a/the detachable connector is configured to seat at a hole through a skull of the patient for coupling the catheter to a/the external tubing
when the catheter is positioned in a skull, a surface of a/the detachable connector is substantially flush with an exterior surface of the skull
a/the target treatment site is an intracranial tumor, a/the fluid includes a therapeutic preparation, and the therapeutic preparation is formulated to slow or inhibit growth of the intracranial tumor
when the target treatment site is in the brain, a burr hole is generated through a skull of the patient, the catheter is delivered through the burr hole and into the brain, and the catheter is maneuvered until it is positioned at the target treatment site in the brain

Various abbreviations may be used herein for standard units, such as deciliter (dl), milliliter (ml), microliter (pi), international unit (IU), centimeter (cm), millimeter (mm), nanometer (nm), inch (in), kilogram (kg), gram (gm), milligram (mg), microgram (μg), millimole (mM), degrees Celsius (° C.), degrees Fahrenheit (° F.), millitorr (mTorr), hour (hr), minute (min), or second (s or sec).

When used in the present disclosure, the terms “e.g.,” “such as”, “for example”, “for an example”, “for another example”, “examples of”, “by way of example”, and “etc.” indicate that a list of one or more non-limiting example(s) precedes or follows; it is to be understood that other examples not listed are also within the scope of the present disclosure.

As used herein, the singular terms “a,” “an,” and “the” may include plural referents unless the context clearly dictates otherwise. Reference to an object in the singular is not intended to mean “one and only one” unless explicitly so stated, but rather “one or more.”

The term “in an embodiment” or a variation thereof (e.g., “in another embodiment” or “in one embodiment”) refers herein to use in one or more embodiments, and in no case limits the scope of the present disclosure to only the embodiment as illustrated and/or described. Accordingly, a component illustrated and/or described herein with respect to an embodiment can be omitted or can be used in another embodiment (e.g., in another embodiment illustrated and described herein, or in another embodiment within the scope of the present disclosure and not illustrated and/or not described herein).

As used herein, the term “set” refers to a collection of one or more objects. Thus, for example, a set of objects can include a single object or multiple objects.

As used herein, the terms “substantially” and “about” are used to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. For example, “substantially” aligned or flush can refer to a range of angular variation of less than or equal to ±10°, such as less than or equal to ±5°, less than or equal to ±4°, less than or equal to ±3°, less than or equal to ±2°, less than or equal to ±1°, less than or equal to ±0.5°, less than or equal to ±0.1°, or less than or equal to ±0.05°.

Additionally, amounts, ratios, and other numerical values may sometimes be presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

The foregoing description of various embodiments of the technology of the present disclosure has been presented for purposes of illustration and description. It is not intended to limit the technology of the present disclosure to the precise forms disclosed. Many modifications, variations and refinements will be apparent to practitioners skilled in the art. For example, embodiments of the device can be sized and otherwise adapted for delivery of therapeutic preparation to other regions of the body. Further, those skilled in the art will recognize, or be able to ascertain using no more than routine experimentation, numerous equivalents to the specific devices and methods described herein. Such equivalents are considered to be within the scope of the present technology of the present disclosure and are covered by the appended claims below.

Elements, characteristics, or acts from one embodiment can be readily recombined or substituted with one or more elements, characteristics or acts from other embodiments to form numerous additional embodiments within the scope of the technology of the present disclosure. Moreover, elements that are shown or described as being combined with other elements, can, in various embodiments, exist as standalone elements. Hence, the scope of the present technology of the present disclosure is not limited to the specifics of the described embodiments, but is instead limited solely by the appended claims.

Although the description herein contains many details, these should not be construed as limiting the scope of the disclosure but as merely providing illustrations of some of the presently preferred embodiments. Therefore, it will be appreciated that the scope of the disclosure fully encompasses other embodiments which may become obvious to those skilled in the art.

All structural and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for”.

What is claimed is:

1. An apparatus for targeted delivery of a therapeutic preparation in a brain of a patient, the apparatus comprising:
a catheter comprising a flexible tube having a proximal end and a distal end, the catheter defining a lumen extending from the proximal end to the distal end, and the tube defining a plurality of slits, wherein the distal end of the tube comprises an atraumatic tip configured to be advanced through an opening in a skull of the patient to a target treatment site within the brain; and
a detachable connector comprising a first coupling member attached to the proximal end of the tube;
wherein the tube is configured such that, upon delivery of a fluid containing the therapeutic preparation to the catheter from an external fluid source, the slits in the tube are responsive to pressure of the fluid within the catheter by the slits opening to disperse the fluid radially outward from the lumen of the tube; and
wherein the tube is further configured such that the slits are responsive to a drop in pressure of the fluid within the catheter, by the slits closing.

2. The apparatus of claim 1, wherein the slits are arranged in a plurality of linear arrays oriented at radially spaced-apart locations along a circumference of the tube to disperse the fluid at multiple radial locations from the tube.

3. The apparatus of claim 1, further comprising an external tubing having a distal end configured for detachably coupling at the detachable connector and a proximal end configured to be coupled to the fluid source.

4. The apparatus of claim 3, wherein when the external tubing is detached from the detachable connector, the detachable connector provides a sealable opening substantially flush with an exterior surface of the skull.

5. The apparatus of claim 1, further comprising:
one or more sleeves configured to be advanced over the tube;
wherein the one or more sleeves each have an inner diameter closely matching an outer diameter of the tube such that when the sleeve is advanced over one or more of the slits, those slits disposed under the sleeve are constrained from opening, thus the apparatus is configured to focus dispersion of fluid through a remainder of the slits which are not constrained by the sleeve.

6. The apparatus of claim 1, further comprising:
a deployable anchor configured to extend from a location within the tube;
wherein upon extension, the anchor is configured to engage tissue for fixation of the tube at the treatment site.

7. The apparatus of claim 1, further comprising:
one or more discs disposed over the tube, wherein the discs are configured to block dispersion of the fluid along the tube past the discs.

8. A system for targeted delivery of a fluid to a brain of a patient, the system comprising:
a catheter comprising a flexible tube having a proximal end and a distal end, the catheter defining a lumen extending from the proximal end to the distal end, the tube defining a plurality of slits, and the tube comprising an atraumatic tip configured to be advanced to a treatment site within the brain;
an external tubing; and
a fluid source configured for delivering a fluid into the catheter via the external tubing;
wherein the tube is configured such that the slits are responsive to pressure applied by the fluid from within the lumen, by the slits opening to disperse the fluid radially outward from the tube; and
wherein the tube is further configured such that the slits are responsive to a drop in pressure within the lumen, by the slits closing to self-seal the lumen.

9. The system of claim 8, further comprising a detachable connector configured to seat at a hole through a skull of the patient for coupling the catheter to the external tubing.

10. The system of claim 8, further comprising a therapeutic preparation disposed in the fluid.

11. The system of claim 8, further comprising:
one or more sleeves disposed over the tube, each sleeve having an inner diameter closely matching an outer diameter of the tube such that one or more slits disposed under the sleeve are constrained from opening, thus configuring the tube to focus dispersion of the fluid to a subset of the slits not disposed under the sleeve.

12. The system of claim 8, further comprising:
a deployable anchor configured to extend from a location within the tube;
wherein the anchor is configured, upon extension, to engage tissue for fixation of the tube at the treatment site.

13. The system of claim 8, further comprising:
one or more discs disposed over the tube, wherein the discs are configured to block dispersion of the fluid along the tube past the discs.

14. The system of claim 8, wherein a target treatment site is an intracranial tumor, the fluid comprises a therapeutic preparation, and the therapeutic preparation is formulated to slow or inhibit growth of the intracranial tumor.

15. A method for targeted delivery of a therapeutic preparation to a brain of a patient, the method comprising:
generating a burr hole through a skull of the patient;
delivering a catheter through the burr hole and into the brain, the catheter comprising a flexible tube having a proximal end and a distal end, the catheter defining a lumen extending from the proximal end to the distal end of the tube, the distal end of the tube comprising an atraumatic tip configured to allow advancement of the tube into the brain, and the tube defining a plurality of slits;
maneuvering the catheter until it is positioned at a target treatment site in the brain;
operating a pump to deliver a fluid containing the therapeutic preparation into the catheter, the delivered fluid causing the slits to expand and dispense fluid from the lumen; and
ceasing delivery of the fluid to the catheter after a period of time, thereby causing a drop in pressure within the catheter such that the slits close and self-seal the tube.

16. The method of claim 15, wherein the slits are disposed in a plurality of linear arrays oriented at radially spaced-apart locations along a circumference of the tube to dispense the fluid from the tube at multiple radial locations of the tube.

17. The method of claim 15, wherein operating a pump to deliver a fluid containing the therapeutic preparation into the catheter comprises attaching an external tubing to the pump and to a detachable connector coupled to the catheter, the method further comprising detaching the external tubing from the detachable connector and covering an opening of the detachable connector.

18. The method of claim 15, further comprising:
advancing one or more sleeves over the tube, wherein each sleeve has an inner diameter closely matching an outer diameter of the tube such that one or more slits disposed under the sleeve are constrained from opening, thus focusing dispersion of the fluid to a subset of slits that are not disposed under the sleeve.

19. The method of claim 15, further comprising:
extending a deployable anchor from a location within the tube; and
engaging the anchor within tissue for fixation of the tube at the treatment site.

20. The method of claim 15, further comprising:
advancing one or more discs over the tube, wherein the discs, when positioned on the tube, act to block dispersion of the fluid along the catheter past the discs.

21. The method of claim 15, wherein a target treatment site is an intracranial tumor, and the therapeutic preparation is formulated to slow or inhibit growth of the intracranial tumor when dispensed at the target treatment site.
